(19) United States
(12) Patent Application Publication
(10) Pub. No.: US 2010/0310465 A1
(43) Pub. Date: Dec. 9, 2010

(54) NANO-DEVICES HAVING RELEASABLE SEALS FOR CONTROLLED RELEASE OF MOLECULES

(75) Inventors: Jeffrey I. Zink, Sherman Oaks, CA (US); Jie Lu, Los Angeles, CA (US); Fuyuhiko Tamanoi, Los Angeles, CA (US); Andre Nel, Sherman Oaks, CA (US); Sarah Angelos, Corvallis, OR (US); Fraser Stoddart, Evanston, IL (US); Qiaolin Chen, Los Angeles, CA (US); Tian Xia, Los Angeles, CA (US); Kaushik Patel, Bayside, WI (US); William Dichtel, Ithaca, NY (US)

Correspondence Address: VENABLE LLP, P.O. BOX 34385, WASHINGTON, DC, 20043-9998, US

(73) Assignee: The Regents of the University of California, Oakland, CA (US)

(21) Appl. No.: 12/841,331

(22) Filed: Jul. 22, 2010

Related U.S. Application Data
(63) Continuation-in-part of application No. PCT/US2009/031891, filed on Jan. 23, 2009.
Application No. PCT/US2009/031891 is a continuation-in-part of application No. PCT/US2009/032451, filed on Jan. 29, 2009.
(60) Provisional application No. 61/006,599, filed on Jan. 23, 2008.
(60) Provisional application No. 61/006,725, filed on Jan. 29, 2008.

(51) Int. Cl. A61K 9/14; A61K 31/352; A61P 27/02; A61P 39/06; A61P 29/00; A61P 37/06; A61P 35/00; A61P 25/08; A61P 31/12; A61P 9/12; A61P 7/02; B01J 19/00; A61K 49/00

(52) U.S. Cl. 424/9.1; 514/454; 424/489; 422/105; 977/724

(57) ABSTRACT

A nanodevice has a containment vessel defining a storage chamber therein and defining at least one port to provide access to and from said storage chamber, and a stopper assembly attached to the containment vessel. The stopper assembly has a blocking unit arranged proximate the at least one port and has a structure suitable to substantially prevent material after being loaded into the storage chamber from being released while the blocking unit is arranged in a blocking configuration. The stopper assembly is responsive to the presence of a predetermined stimulus such that the blocking unit is released in the presence of the predetermined stimulus to allow the material to be released from the storage chamber. The predetermined stimulus is a predetermined catalytic activity that is suitable to at least one of cleave, hydrolyze, oxidize, or reduce a portion of the stopper assembly, and the nanodevice has a maximum dimension of about 1 μm.

### CROSS-REFERENCE TO RELATED APPLICATIONS

This application is a continuation-in-part application that claims priority to International Patent Application No. PCT/US2009/031891 filed Jan. 23, 2009, which claims priority to U.S. Provisional Application No. 61/006,599 filed Jan. 23, 2008, and claims priority to International Patent Application No. PCT/US2009/032451 filed Jan. 29, 2009, which claims priority to and U.S. Provisional Application No. 61/006,725 filed Jan. 29, 2008, the entire contents of all of which are hereby incorporated by reference in entirety.

### FEDERAL FUNDING INFORMATION

This invention was made with U.S. Government support of Grant Nos. CHE 0507929 and DMR 0346601, awarded by the National Science Foundation, and of Grant No. 32737, awarded by NIH. The U.S. Government has certain rights in this invention.

### BACKGROUND

1. Field of Invention

The current invention relates to nano-devices, and more specifically to nano-nano-devices that have releasable seals for controlled release of molecules contained therein.

2. Discussion of Related Art

Control of molecular transport in, through, and out of mesopores has important potential applications in nanoscience including fluidics and drug delivery. Surfactant-templated silica (Kresge, C. T.; Leonowicz, M. E.; Roth, W. J.; Vartuli, J. C.; Beck, J. S, Nature 1992, 359, 710-712) is a versatile material in which ordered arrays of mesopores can be easily synthesized, providing a convenient platform for attaching molecules that undergo large amplitude motions to control transport. Mesostructured silica is transparent (for photocontrol and spectroscopic monitoring), and can be fabricated into useful morphologies (thin films (Lu, Y. F.; Ganguli, R.; Drewien, C. A.; Anderson, M. T.; Brinker, C. J.; Gong, W. L.; Guo, Y. X.; Soyez, H.; Dunn, B.; Huang, M. H.; Zink, J. I. Nature 1997, 389, 364-368), particles (Kresge, C. T.; Leonowicz, M. E.; Roth, W. J.; Vartuli, J. C.; Beck, J. S, Nature 1992, 359, 710-712; Huh, S.; Wiench, J. W.; Yoo, J. C.; Pruski, M.; Lin, V. S. Y. Chem. Mater. 2003, 15, 4247-4256)) with designed pore sizes and structures.

Mesoporous silica nanoparticles coated with molecular valves hold the promise to encapsulate a payload of therapeutic compounds, to transport them to specific locations in the body, and to release them in response to either external or cellular stimuli. Sequestering drug molecules serves the dual purpose of protecting the payload from enzymatic degradation, while reducing the undesired side-effects associated with many drugs. Although these benefits are common to pro-drug strategies ((a) Hirano, T.; Klesse, W.; Ringsdorf, H. Makromol. Chem. 1979, 180, 1125. (b) Kataoka, K.; Harada, A.; Nagasaki, Y. Adv. Drug Delivery Rev. 2001, 47, 113. (c) Padilla De Jesus, O. L.; Ihre H. R.; Gagne, L.; Frechet, J. M. J.; Szoka, F. C. Jr. Bioconjug Chem. 2002, 13, 453. (d) Denny, W. A. Cancer Invest. 2004, 22, 604. (e) Lee, C. C.; MacKay, J. A.; Frechet, J. M. J., et al. Nat. Biotechnol. 2005, 23, 1517. (f) Duncan, R.; Ringsdorf, H.; Satchi-Fainaro, R. J. Drug Target. 2006, 14,337. (g) Tietze, L. F.; Major, F.; Schuberth, I. Angew. Chem. Int. Ed. 2006, 45, 6574), the nanoparticle-supported nanovalve system does not require covalent modification of the therapeutic compounds and allows for the release of many drug molecules upon each stimulus event ((a) Duncan, R.; Vicent, M. J.; Greco, F., et al. Endocr-Relat. Cancer. 2005, 12, 5189. (b) Pantos, A.; Tsiourvas, D.; Nounesis, G.; Paleos, C. M. Langmuir 2005, 21, 7483. (c) Dhanikula, R. S.; Hildgen, P. Bioconjug. Chem. 2006, 17, 29. (d) Darbre, T.; Reymond, J.-L. Acc. Chem. Res. 2006, 39, 925. (e) Gopin, A.; Ebner, S.; Attali, B.; Shabat, D. Bioconjug. Chem. 2006, 17, 1432). Recently, it was demonstrated that mesoporous silica nanoparticles, not modified with molecular machinery, can deliver the water-insoluble drug camptothecin into human pancreatic cancer cells with very high efficiency (Lu, J. Liong, M.; Zink, J. I.; Tamanoi, F. Small 2007, 3, 1341). For more sophisticated drug delivery applications, the ability to functionalize ((a) Hernandez, R.; Tseng, H.-R.; Wong, J. W.; Stoddart, J. F.; Zink, J. I. J. Am. Chem. Soc. 2004, 126, 3370. (b) Nguyen, T. D.; Tseng, H.-R.; Celestre, P. C.; Flood, A. H.; Liu, Y.; Stoddart, J. F.; Zink, J. I. Proc. Natl. Acad. Sci. U.S.A. 2005, 102, 10029. (c) Nguyen, T. D.; Leung, K. C.-F.; Liong, M.; Pentecost, C. D.; Stoddart, J. F.; Zink, J. I. Org. Lett. 2006, 8, 3363. (d) Leung, K. C.-F.; Nguyen, T. D.; Stoddart, J. F.; Zink, J. I. Chem. Mater. 2006, 18, 5919. (e) Nguyen, T. D.; Liu, Y.; Saha, S.; Leung, K. C.-F.; Stoddart, J. F.; Zink, J. I. J. Am. Chem. Soc. 2007, 129, 626. (f) Nguyen, T. D.; Leung, K. C. F.; Liong, M.; Liu, Y.; Stoddart, J. F.; Zink, J. I. Adv. Funct. Mater. 2007, 17, 2101. (g) Saha, S.; Leung, K. C. F.; Nguyen, T. D.; Stoddart, J. F.; Zink, J. I. Adv. Funct. Mater. 2007, 17, 685. (h) Angelos, S.; Johansson, E.; Stoddart, J. F.; Zink, J. I. Adv. Funct. Mater. 2007, ASAP article) nanoparticles with nanovalves and other controlled-release mechanisms has become an area of widespread interest ((a) Mal, N. K.; Fujiwara, M.; Tanaka, Y.; Nature 2003, 421, 350. (b) Giri, S.; Trewyn, B. G.; Stellmaker, M. P.; Lin, V. S. Y. Angew. Chem. Int. Ed. 2005, 44, 5038. (c) Kocer, A.; Walko, M.; Meijberg, W.; Feringa, B. L. Science 2005, 309, 755. (d) Angelos, S.; Choi, E.; Vogtle, F.; De Cola, L.; Zink, J. I. J. Phys. Chem. C 2007, 111, 6589. (e) Slowing, I.; Trewyn, B. G.; Giri, S.; Lin, V. S. Y. Adv. Funct. Mater. 2007, 17, 1225). Previously, we have demonstrated the operation of molecular and supramolecular valves in non-biologically relevant contexts using redox (Hernandez, R.; Tseng, H.-R.; Wong, J. W.; Stoddart, J. F.; Zink, J. I. J. Am. Chem. Soc. 2004, 126, 3370. Nguyen, T. D.; Tseng, H.-R.; Celestre, P. C.; Flood, A. H.; Liu, Y.; Stoddart, J. F.; Zink, J. I. Proc. Natl. Acad. Sci. U.S.A. 2005, 102, 10029. Nguyen, T. D.; Liu, Y.; Saha, S.; Leung, K. C.-F.; Stoddart, J. F.; Zink, J. I. J. Am. Chem. Soc. 2007, 129, 626.), pH (Nguyen, T. D.; Leung, K. C.-F.; Liong, M.; Pentecost, C. D.; Stoddart, J. F.; Zink, J. I. Org. Lett. 2006, 8, 3363.), competitive binding (Leung, K. C.-F.; Nguyen, T. D.; Stoddart, J. F.; Zink, J. I. Chem. Mater. 2006, 18, 5919.), and light (Nguyen, T. D.; Leung, K. C. F.; Liong, M.; Liu, Y.; Stoddart, J. F.; Zink, J. I. Adv. Funct. Mater. 2007, 17, 2101.) as actuators. Other controlled release systems include photoresponsive azobenzene-based nanoimpellers (Angelos, S.; Choi, E.; Vogtle, F.; De Cola, L.; Zink, J. I. J. Phys. Chem. C 2007, 111, 6589.), chemically removable CdS nanoparticle caps (Giri, S.; Trewyn, B. G.; Stellmaker, M. P.; Lin, V. S. Y. Angew. Chem. Int. Ed. 2005, 44, 5038. Slowing, I.; Trewyn, B. G.; Giri, S.; Lin, V. S. Y. Adv. Funct. Mater. 2007, 17, 1225.), and reversible photo-dimerization of tethered coumarins (Mal, N. K.; Fujiwara, M.; Tanaka, Y.; Nature 2003, 421, 350.).

Although there has been substantial research activity in this field, there still remains a need for suitable nano-devices that can selectively release molecules from a containment vessel and that can also keep the molecules substantially contained within the containment vessel when not being selectively released. There further remains a need for such nano-devices that can be useful for biological and biomedical applications.

### SUMMARY

A nanodevice according to some embodiments of the current invention has a containment vessel defining a storage chamber therein and defining at least one port to provide access to and from said storage chamber, and a stopper assembly attached to the containment vessel. The stopper assembly has a blocking unit arranged proximate the at least one port and has a structure suitable to substantially prevent material after being loaded into the storage chamber from being released while the blocking unit is arranged in a blocking configuration. The stopper assembly is responsive to the presence of a predetermined stimulus such that the blocking unit is released in the presence of the predetermined stimulus to allow the material to be released from the storage chamber. The predetermined stimulus is a predetermined catalytic activity that is suitable to at least one of cleave, hydrolyze, oxidize, or reduce a portion of the stopper assembly, and the nanodevice has a maximum dimension of about 1 μm.

A composition of matter according to some embodiments of the current invention has a plurality of nanoparticles, each defining a storage chamber therein; and a guest material contained within the storage chambers defined by the plurality of nanoparticles. The guest material is substantially chemically non-reactive with the nanoparticles. The plurality of nanoparticles are operable to cause the guest material contained within the storage chambers to be released in a presence of a predetermined stimulus, and each nanoparticle of the plurality of nanoparticles has a maximum dimension of about 1 μm.

A method of administering at least one of a biologically active substance or a diagnostic substance according to some embodiments of the current invention includes administering a composition to at least one of a person, animal, or organism, the composition comprising nanoparticles therein, wherein the nanoparticles contain the at least one of a biologically active substance or an imaging/tracking substance therein; and at least one of directing or allowing the nanoparticles of the administered composition to come into contact with a predetermined catalytic activity that is suitable to at least one of cleave, hydrolyze, oxidize, or reduce a portion of the nanoparticles to release the biologically active substance or the imaging/tracking substance from the nanoparticles.

### BRIEF DESCRIPTION OF THE DRAWINGS

Further objectives and advantages will become apparent from a consideration of the description, drawings, and examples.

FIG. 1A is a schematic illustration of a nano-device according to an embodiment of the current invention.

FIG. 1B is a schematic illustration of a nano-device, and methods of production, that can serve as a precursor according to some embodiments of the current invention.

FIG. 2 is schematic illustration to help explain additional embodiments of the current invention.

FIG. 3 is a schematic illustration of two embodiments of the current invention that have different stoppers.

FIG. 4 shows emission intensity plot (λ_{cx} 514 nm) of HEPES buffer solutions (50 mM, pH=7.5) containing ester (green) or amide (blue) stoppered snap-tops corresponding to FIG. 3. The response of the ester system to the deactivated enzyme (red) is also shown.

FIG. 5 illustrates an example of a mechanism for chemically attaching stoppers to nanodevices according to some embodiments of the current invention.

FIG. 6 summarizes some examples of stoppers according to some embodiments of the current invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited herein are incorporated by reference as if each had been individually incorporated.

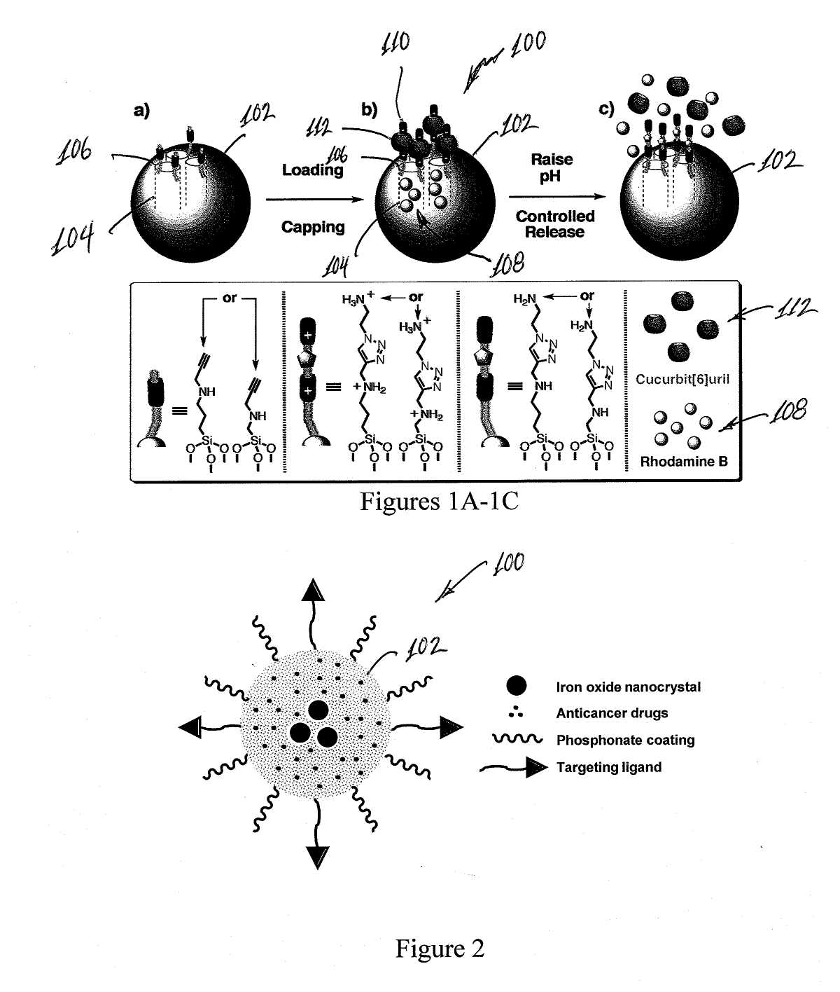

FIG. 1A is a schematic illustration of a nanodevice 100 according to an embodiment of the current invention. The nanodevice 100 has a containment vessel 102 defining a storage chamber 104 therein and defining at least one port 106 to provide access for the transfer of material 108 into and/or out of the storage chamber 104. The containment vessel 102 can be a mesoporous silica nanoparticle in some embodiments of the current invention. The material 108 can be molecules which are sometimes also referred to as guest molecules herein. However, the material 108 does not always have to be in the form of molecules in some embodiments of the current invention. The material 108 is also referred to as cargo herein since it can be loaded into the nanodevice 100. As is indicated in FIG. 1A, the nanodevice can be referred to as a Snap-Top Covered Silica Nanocontainer (SCSN) in some embodiments of the current invention. The nanodevice 100 also has a stopper assembly 110 attached to said containment vessel 102. The stopper assembly 110 has a blocking unit 112 arranged proximate the at least one port 106 and has a structure suitable to substantially prevent material 108 after being loaded into said storage chamber 104 from being released while the blocking unit 112 is arranged in a blocking configuration. The stopper assembly 110 is responsive to the presence of a predetermined stimulus such that the blocking unit 112 is released in the presence of the predetermined stimulus to allow the material 108 to be released from the storage chamber 106. The predetermined stimulus can be a predetermined catalytic activity, for example, that is suitable to at least one of cleave, hydrolyze, oxidize, or reduce a portion of the stopper assembly 110.

The nanodevice 100 has a maximum dimension of less than about 1 μm and greater than about 50 nm in some embodiments. For some embodiments, the nanodevice 100 has a maximum dimension of less than about 400 nm and greater than about 50 nm. When the nanodevice 100 is greater than about 400 nm, it becomes too large to enter into biological cells. On the other hand, when the nanodevice 100 is less than about 50 nm, it becomes less able to contain a useful number of molecules therein. Furthermore, when the nanodevices are less than about 300 nm, they become more useful in some applications to biological systems. For some embodiments of the current invention, nanodevices having a maximum dimension in the range of about 50 nm to about 150 nm are suitable. The containment vessel can be, but is not limited to, a mesoporous silica nanoparticle according to some embodiments of the current invention.

In some embodiments of the current invention, the stopper assembly 110 can include a thread 114 onto which the blocking unit 112 can be threaded as is illustrated schematically in FIG. 1A. The thread 114 has a longitudinal length that is long relative to a transverse length and is suitable to be attached at one longitudinal end to the containment vessel 102. The stopper assembly 110 can also have a stopper 116 attached to a second longitudinal end of the thread 114 in some embodiments of the current invention. According to some aspects of the current invention, the stopper 116 can be selected among a wide range of possible stoppers based on the type of environment for which the material 108 will be released.

In operation, the blocking unit 112 of the stopper assembly 110 is held in place at the port 106 by the thread 114 and the stopper 116 according to some embodiments of the current invention. The stopper 116 is selected to respond to a stimulus so that it allows the blocking unit 112 to move away from the port 106. The stimulus can be an environmental condition such as a local chemical environment or can be an applied condition such as illumination with light, etc. The stopper 116 can be cleaved, for example, from the thread 114 by an environmental condition according to some embodiments of the current invention. Once the blocking unit 112 is released to move away from the port 106, the material 108 can then escape from the storage chamber 104.

According to some embodiments of the current invention, a synthetic strategy can involve the use of a snap-top “precursor”. The nanodevice 100 with the stopper 116 can serve as a precursor according to some embodiments of the current invention. The assembly of the snap-top precursors can be performed step-wise from the silica nanoparticle surfaces outward according to an embodiment of the current invention, as illustrated in FIG. 1B. However, the general concepts of the current invention are not limited to only the materials used in the example of FIG. 1B. First, the silica nanoparticles are treated with aminopropyltriethoxysilane (APTES) to achieve an amine-modified nanoparticle surface. An azideterminated tri(ethylene)glycol thread is attached to the amine-modified nanoparticles, and the pores are then loaded by soaking in a concentrated cargo solution and allowing the cargo to diffuse into the empty pores. The precursor is completed through the addition of α-cyclodextrin as the blocking unit at 5° C., which complexes with the threads at the low temperature. The precursor can enable the preparation of many different systems based on a common general structure in which different stoppers can be attached depending on the specific desired application according to some embodiments of the current invention.

The material or molecules of interest to be stored in and released from the containment vessels 102 can include, but are not limited to, biologically active substances. The term “biologically active substance” as used herein is intended to include all compositions of matter that can cause a desired effect on biological material or a biological system and may include in situ and in vivo biological materials and systems. The biologically active substance may be selected from such substances that have molecular sizes such that they can be loaded into the nanodevices, and can also be selected from such substances that don't react with the nanodevices. A biological system may include a person, animal or plant, for example.

Biologically active substances may include, but are not limited to, the following:

(1) Small molecule drugs for anticancer treatment such as camptothecin, paclitaxel and doxorubicin;

(2) Ophthalmic drugs such as flurbiprofen, levobbunolol and neomycin;

(3) Nucleic acid reagents such as siRNA and DNAzymes;

(4) Small molecule antioxidants such as n-acetylcysteine, sulfurophane, vitamin E, vitamin C, etc.;

(5) Small molecule drugs for immune suppression such as rapamycin, FK506, cyclosporine; and

(6) Any pharmacological compound that can fit into the nanodevice, e.g., analgesics, NSAIDS, steroids, hormones, anti-epileptics, anti-arrythmics, anti-hypentensives, antibiotics, antiviral agents, anticoagulants, platelet drugs, cardiostimulants, cholesterol lowering agents, etc.

Molecules of interest can also include imaging and/or tracking substances. Imaging and/or tracking substances may include, but are not limited to, dye molecules such as propidium iodide, fluorescein, rhodamine, green fluorescent protein and derivatives thereof.

FIG. 2 is a schematic illustration to facilitate the explanation of additional embodiments of the current invention. For the sake of clarity, FIG. 2 does not show storage chambers, such as a plurality of pores of a mesoporous silica nanoparticle, and does not show stopper assemblies. However, it should be understood that they can be present in addition to the features illustrated in FIG. 2. According to some embodiments of the current invention, the nanodevices, such as nanodevice 100, can include a plurality of anionic molecules attached to the surface of the nanodevice as is illustrated schematically in FIG. 2. For example the anionic molecules can be phosphonate moieties attached to the outer surface of the nanodevice to effectively provide a phosphonate coating on the nanodevice. For example, the anionic molecules can be trihydroxysilylpropyl methylphosphonate molecules according to an embodiment of the current invention.

A phosphonate coating on the containment vessel, such as containment vessel 102, can provide an important role in some biological applications according to some embodiments of the current invention. This phosphonate coating can provide a negative zeta potential that is responsible for electrostatic repulsion to keep such submicron structures dispersed in an aqueous tissue culture medium, for example. This dispersion can also be important for keeping the particle size limited to a size scale that allows endocytic uptake (i.e., hinders clumping). In addition to size considerations, the negative zeta potential may play a role in the formation of a protein corona on the particle surface that can further assist cellular uptake in some applications. It is possible that this could include molecules such as albumin, transferrin or other serum proteins that could participate in receptor-mediated uptake. In addition to the role of the phosphonate coating for drug delivery, it can also provide beneficial effects for molecule loading according to some embodiments of the current invention. (See co-pending application number PCT/U.S.08/13476, co-owned by the assignee of the current application, the entire contents of which are incorporated by reference herein.)

The nanodevice 100 can also be functionalized with molecules in additional to anionic molecules according to some embodiments of the current invention. For example, a plurality of folate ligands can be attached to the outer surface of the containment vessel 102 according to some embodiments of the current invention, as is illustrated schematically in FIG. 2 (stopper assemblies are not shown for clarity).

In some embodiments of the current invention, the nanodevice 100 can also include fluorescent molecules contained in or attached to the containment vessel 102. For example, fluorescent molecules may be attached inside the pores of mesoporous silica nanoparticles according to some embodiments of the current invention. For example, the fluorescent molecules can be an amine-reactive fluorescent dye attached by being conjugated with an amine-functionalized silane according to some embodiments of the current invention. Examples of some fluorescent molecules, without limitation, can include fluorescein isothiocyanate, NHS-fluorescein, rhodamine β isothiocyanate, tetramethylrhodamine β isothiocyanate, and/or Cy5.5 NHS ester.

In further embodiments of the current invention, the nanodevices 100 may further comprise one or more nanoparticle of magnetic material formed within the containment vessel 102, as is illustrated schematically in FIG. 2 for one particular embodiment. For example, the nanoparticles of magnetic material can be iron oxide nanoparticles according to an embodiment of the current invention. However, the broad concepts of the current invention are not limited to only iron oxide materials for the magnetic nanoparticles. Such nanoparticles of magnetic material incorporated in the submicron structures can permit them to be tracked by magnetic resonance imaging (MRI) systems and/or manipulated magnetically, for example.

In further embodiments of the current invention, the nanodevices 100 may further comprise one or more nanoparticle of a material that is optically dense to x-rays. For example, gold nanoparticles may be formed within the containment vessel 102 of the nanodevice 100 according to some embodiments of the current invention.

### Examples

In the following example, we describe the design, synthesis, and operation of a novel, biocompatible controlled release motif we call snap-top covered silica nanocontainers (SCSNs), based on an embodiment of the current invention. This is an example of a nanodevice according to an embodiment of the current invention in which the “snap-top” assembly corresponds to a stopper assembly. Silica nanoparticles (˜400 nm in diameter) that contain hexagonally arranged pores (˜2 nm diameter) function as both the snap-top supports and as containers for guest molecules. The porous mesostructure ((a) Kresge, C. T.; Leonowicz, M. E.; Roth, W. J.; Vartuli, J. C.; Beck, J. S, Nature 1992, 359, 710. (b)) Lu, Y. F.; Ganguli, R.; Drewien, C. A.; Anderson, M. T.; Brinker, C. J.; Gong, W. L.; Guo, Y. X.; Soyez, H.; Dunn, B.; Huang, M. H.; Zink, J. I. Nature 1997, 389, 364. (c) Huang, M. H.; Dunn, B. S.; Soyez, H.; Zink, J. I. Langmuir 1998, 14, 7331) is templated by cetyltrimethylammonium bromide (CTAB) surfactants, and particle synthesis is accomplished using a base-catalyzed sol-gel procedure (Huh, S.; Wiench, J. W.; Yoo, J. C.; Pruski, M.; Lin, V. S. Y. Chem. Mater. 2003, 15, 4247). Methods for derivatizing silica are well-known ((a) Hernandez, R.; Franville, A. C.; Minoofar, P.; Dunn, B.; Zink, J. I. J. Am. Chem. Soc. 2001, 123, 1248. (b) Minoofar, P. N.; Hernandez, R.; Chia, S.; Dunn, B.; Zink, J. I.; Franville, A. C. J. Am. Chem. Soc. 2002, 124, 14388. (c) Minoofar, P. N.; Dunn, B. S.; Zink, J. I. J. Am. Chem. Soc. 2005, 127, 2656) and are used here to functionalize the nanoparticle surfaces with the snap-top machinery. In general, a snap-top consists of a [2]rotaxane tethered to the surface of a nanoparticle in which an α-cyclodextrin (α-CD) tori encircles a polyethylene glycol thread and is held in place by a cleavable stopper. When closed, the snap-top contains guest molecules stored within the pores, but releases the guests following cleavage of the stopper and dethreading of the tori. Based on the design of the stopper, we conceive that a multitude of stimuli could be exploited to activate snap-top systems. The specific snap-top system we describe here in this example releases encapsulated cargo molecules following enzyme-mediated hydrolysis.

We have taken divergent approaches in the design and synthesis of SCSNs in which the use of a single versatile snap-top precursor that can enable the preparation of multiple systems that are ultimately highly specific and differentiated in their function. In the divergent design, a snap-top precursor having an unstoppered [2]pseudorotaxanes serves as a foundation from which various snap-top systems can be created depending on the specific stopper that is attached. The synthesis of the snap-top precursor is carried out in a step-wise fashion from the nanoparticle surface outward (FIG. 1). The mesoporous silica is first treated with aminopropyltriethoxysilane to achieve an amine-modified surface. The amine-functionalized material is then alkylated with a triethyleneglycol monoazide monotosylate unit to give an azide-terminated surface. Cargo molecules are loaded into the nanopores by diffusion, and the loaded, azide-modified particles are then incubated with α-CD at 5° C. for 24 h. The α-CD tori thread onto the triethyleneglycol chains at low temperature effectively blocking the nanopores, while the azide function serves as a handle to attach a stoppering group. The stoppers are chemically attached to the snap-top precursors using the Cu(I)-catalyzed azide-alkyne cycloaddition ((a) Rostovtsev, V. V.; Green, L. G.; Fokin, V. V.; Sharpless, K. B. Angew. Chem., Int. Ed. 2002, 41, 2596. (b) Tomoe, C. W.; Christensen, C.; Meldal, M. J. Org. Chem. 2002, 67, 3057), a transformation chosen because of its remarkable functional group tolerance and high efficiency as well as our recent success in utilizing it for the preparation of interlocked molecules ((a) Dichtel, W. R.; Miljanic, O, S.; Spruell, J. M.; Heath, J. R.; Stoddart, J. F. J. Am. Chem. Soc. 2006, 128, 10388. (b) Miljanic, O, S.; Dichtel, W. R.; Mortezaei, S.; Stoddart. J. F. Org. Lett. 2006, 8, 4835. (c) Aprahamian, I.; Dichtel, W. R.; Ikeda, T.; Heath, J. R.; Stoddart, J. F. Org. Lett. 2007, 9, 1287. (d) Braunschweig, A. B.; Dichtel, W. R.; Miljanic, O, S.; Olson, M. A.; Spruell, J. M.; Khan, S. I.; Heath, J. R.; Stoddart, J. F. Chem. Asian J. 2007, 2, 634).

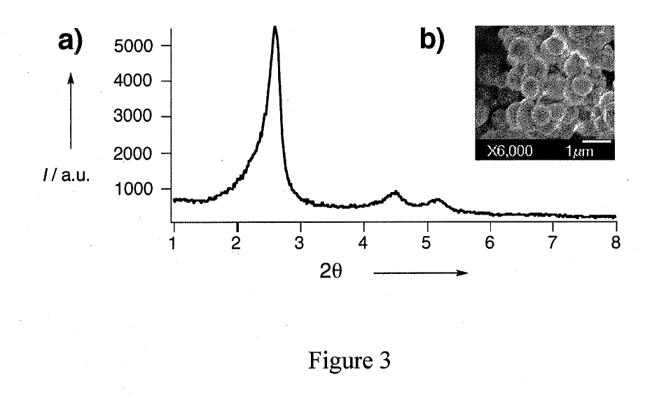

To test the viability of an enzyme-responsive snap-top motif, a system activated by Porcine Liver Esterase (PLE) (Woodroofe, C. C.; Lippard, S. J. J. Am. Chem. Soc. 2003, 125, 11458) was designed (FIG. 3). To prepare a PLE-responsive SCSN, a precursor loaded with luminescent cargo molecules (Rhodamine B) was capped with the ester-linked adamantyl stopper 2a. In this snap-top system, PLE catalyzes the hydrolysis of the adamantyl ester stopper, resulting in dethreading of the α-CD, and release of the cargo molecules from the pores. As a control, an SCSN was also prepared using the adamantyl amide analog 2b, which does not undergo hydrolysis by PLE. After the stoppering reactions, the dye-loaded silica particles were filtered and washed to remove non-specifically adsorbed small contaminants.

The successful functionalization of the nanoparticle surface was confirmed by FT-IR spectroscopy at various stages of loading and release. For the azide-modified nanoparticles, the peak at 3450 cm⁻¹ is indicative of an N—H stretch while a strong absorption between 1050 cm⁻¹ and 1300 cm⁻¹ indicates the presence of different kinds of C—N bonds. The control amide snap-top system shows two distinctive absorption peaks for the amide C═O group at 1650 cm⁻¹ and 1600 cm⁻¹. The ester-functionalized snap-top system shows instead the expected ester C═O stretch at 1731 cm⁻¹ with pronounced C—H absorptions arising from the adamantyl group. In the spectra of the nanoparticles after guest release, the region around 3000 cm⁻¹ is broad, a feature which is characteristic of the new carboxylic acid functionality while the C═O peak is still evident at 1731 cm⁻¹ indicating some remaining ester functionalities on the surface of the nanoparticles.

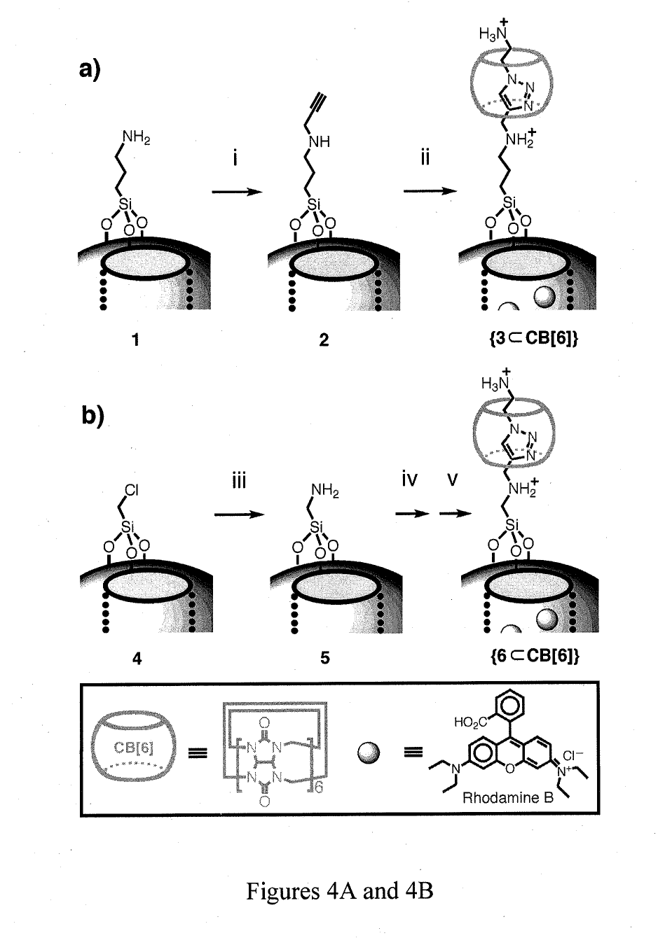

The enzyme-triggered release of cargo molecules was monitored using luminescence spectroscopy. The dye-loaded, stoppered particles (15 mg) were placed into the corner of a cuvette before carefully adding HEPES buffer (50 mM, 12 mL, pH=7.5). To open the snap-tops, a solution of PLE [0.12 mL, 10 mg/mL in 3.2 M (NH₄)₂SO₄] was carefully added while the solution was stirred. The emission of Rhodamine B in the solution above the particles was measured as a function of time using a 514 nm probe beam (15 mW), both before and after addition of PLE (FIG. 4).

Prior to the addition of PLE, the emission intensity of Rhodamine B is essentially constant, indicating that the dye remains trapped in the pores of the silica particles. The emission intensity begins to increase almost immediately following addition of PLE. The emission intensity asymptotically approaches its maximum value with a half-life of ˜5 min. By contrast, no such increase in emission was observed for the amide-stoppered snap-top system. In order to further demonstrate that the enzyme is responsible for the release, it was denatured by heating at 50° C. for 30 mins before addition to the ester-stoppered snap-tops. No release of dye was observed. Taken together, these results are consistent with the specific opening of the snap-tops as a result of the enzyme-mediated hydrolysis of the adamantyl ester stoppers.

In order to estimate the payload of molecules that are released by the snap-top system, the absorbance of the solution above the particles was measured before and after release. Using these data, it was calculated that for 15 mg of particles, 0.45 μmol (1.4 wt %) of Rhodamine B is released.

Described herein is a versatile system that is capable of entrapment and controlled release of cargo molecules. We have used one snap-top precursor to prepare two different snap-top systems, one with an ester-linked stopper, and the other with an amide-linked stopper. Using luminescence spectroscopy, we have demonstrated the ability of PLE to selectively activate the ester-linked snap-top system while the amide-linked system is left intact. The can provide a biocompatible controlled release system that exploits enzymatic specificity according to some embodiments of the current invention. Because of the wide range of stoppering units that could be attached to the SCSN precursor, a multitude of snap-top systems with differentiated modes of activation could be prepared with relative ease. In the future, the divergent synthetic approach that we have described will allow the snap-top motif to be very easily adapted to accommodate many different applications.

### Further Snap Top Embodiments

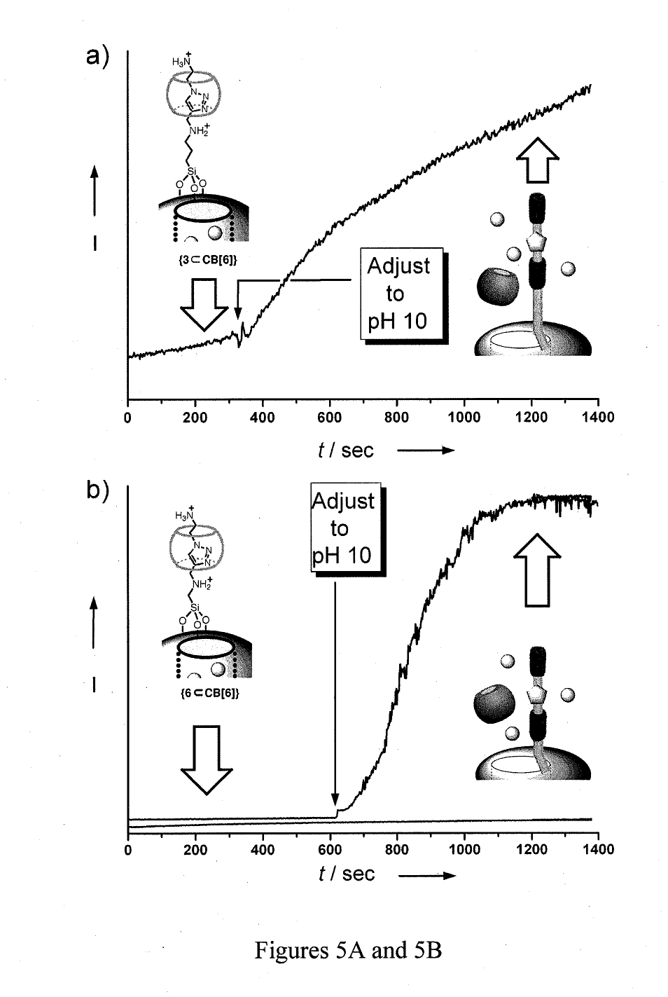

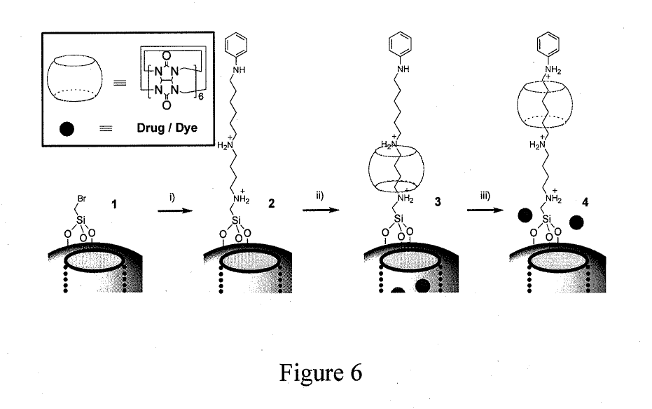

The reactivity of a given snap-top system can be determined by the specific stopper that is attached (FIG. 5) to the snap-top precursor. The azide function of the precursors can serve as a handle to attach a stoppering group. Stoppers can be attached through Cu(I)-catalyzed azide-alkyne cycloaddition (‘Click’ Chemistry). FIG. 6 shows three different stoppers according to some embodiments of the current invention that respond to enzymes, pH, and redox stimulation. However, the broad concepts of the current invention are not limited to only these specific examples. There are a wide range of possible stoppers that may be selected according to the particular application.

In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A nanodevice, comprising:
a containment vessel defining a storage chamber therein and defining at least one port to provide access to and from said storage chamber; and
a stopper assembly attached to said containment vessel, said stopper assembly comprising a blocking unit arranged proximate said at least one port and having a structure suitable to substantially prevent material after being loaded into said storage chamber from being released while said blocking unit is arranged in a blocking configuration,
wherein said stopper assembly is responsive to the presence of a predetermined stimulus such that said blocking unit is released in the presence of said predetermined stimulus to allow said material to be released from said storage chamber,
wherein said predetermined stimulus is a predetermined catalytic activity that is suitable to at least one of cleave, hydrolyze, oxidize, or reduce a portion of said stopper assembly, and
wherein said nanodevice has a maximum dimension of about 1 μm.

2. A nanodevice according to claim 1, wherein said nanodevice has a maximum dimension of less than about 400 nm and greater than about 50 nm.

3. A nanodevice according to claim 1, wherein said nanodevice has a maximum dimension of less than about 300 nm and greater than about 50 nm.

4. A nanodevice according to claim 1, wherein said nanodevice has a maximum dimension of less than about 150 nm and greater than about 50 nm.

5. A nanodevice according to claim 1, further comprising a thread attached to said containment vessel proximate said port, said blocking unit having a structure so that it can become threaded over said thread.

6. A nanodevice according to claim 5, further comprising a stopper attached to said thread such that said stopper at least assists in holding said blocking unit in said blocking configuration.

7. A nanodevice according to claim 1, wherein said nanodevice is operable in an aqueous environment.

8. A nanodevice according to claim 1, wherein said nanodevice consists essentially of biocompatible materials in a composition thereof.

9. A nanodevice according to claim 1, wherein said containment vessel comprises silica in a material thereof.

10. A nanodevice according to claim 9, wherein said containment vessel is a mesoporous silica nanoparticle defining a plurality of substantially parallel pores therein, said storage chamber being one of said plurality of substantially parallel pores.

11. A nanodevice according to claim 1, wherein said stopper assembly comprises at least one of a [2]rotaxane or a [2]pseudorotaxane macromolecule.

12. A nanodevice according to claim 11, wherein said blocking unit of said stopper assembly is an α-cyclodextrin toroidal molecule.

13. A nanodevice according to claim 12, wherein said stopper assembly comprises a polyethylene thread attached to said containment vessel.

14. A nanodevice according to claim 13, wherein said stopper assembly further comprises a stopper attached to said polyethylene thread, said stopper being responsive to said predetermined stimulus to release said blocking unit, wherein said stopper is suitable to hold said blocking unit in said blocking configuration prior to being exposed to said predetermined stimulus.

15. A nanodevice according to claim 1, further comprising a plurality of anionic or electrostatic molecules attached to an outer surface of said containment vessel,
wherein said anionic or electrostatic molecules provide hydrophilicity or aqueous dispersability to said nanodevice and are suitable to provide repulsion between other similar nanodevices.

16. A nanodevice according to claim 15, wherein said anionic molecules comprise a phosphonate moiety.

17. A nanodevice according to claim 15, wherein said plurality of anionic molecules are trihydroxysilylpropyl methylphosphonate.

18. A nanodevice according to claim 1, further comprising folate ligands attached to said containment vessel.

19. A nanodevice according to claim 1, further comprising a nanoparticle of magnetic material formed within said containment vessel of said nanodevice.

20. A nanodevice according to claim 19, wherein said nanoparticle of magnetic material is an iron oxide nanoparticle.

21. A nanodevice according to claim 1, further comprising a nanoparticle of gold formed within said containment vessel of said nanodevice.

22. A composition of matter, comprising:
a plurality of nanoparticles, each defining a storage chamber therein; and
a guest material contained within said storage chambers defined by said plurality of nanoparticles, said guest material being substantially chemically non-reactive with said nanoparticles,
wherein said plurality of nanoparticles are operable to cause said guest material contained within said storage chambers to be released in a presence of a predetermined stimulus, and
wherein each nanoparticle of said plurality of nanoparticles has a maximum dimension of about 1 μm.

23. A composition of matter according to claim 22, wherein said release in the presence of said predetermined stimulus comprises a predetermined enzyme cleaving a portion of a stopper assembly to release a stopper.

24. A composition according to claim 22, wherein said plurality of nanoparticles are each mesoporous silica nanoparticles, each defining a plurality of substantially parallel pores therein, said storage chambers each being a respective one of said plurality of substantially parallel pores.

25. A composition according to claim 22, wherein said stopper assembly comprises at least one of a [2]rotaxane or a [2]pseudorotaxane macromolecule.

26. A composition according to claim 22, wherein said blocking unit is an α-cyclodextrin toroidal molecule.

27. A composition according to claim 22, wherein said stopper assembly comprises a polyethylene thread attached to said containment vessel.

28. A composition according to claim 22, further comprising a hydrophilic silane.

29. A composition according to claim 22, further comprising folate.

30. A composition according to claim 22, further comprising a ligand for targeting a specific cell, a specific tissue, specific organ or specific biological component.

31. A method of administering at least one of a biologically active substance, a therapeutic substance, a neutraceutical substance, a cosmetic substance or a diagnostic substance, comprising:
administering a composition to at least one of a person, animal, plant, or organism, said composition comprising nanoparticles therein, wherein said nanoparticles contain said at least one of a biologically active substance or an imaging/tracking substance therein; and
at least one of directing or allowing said nanoparticles of said administered composition to come into contact with a predetermined catalytic activity that is suitable to at least one of cleave, hydrolyze, oxidize, or reduce a portion of said nanoparticles to release said substance from said nanoparticles.

32. A nanodevice, comprising:
a containment vessel defining a storage chamber therein and defining at least one port to provide transfer of matter to or from said storage chamber; and
a valve assembly attached to said containment vessel;
wherein said valve assembly is operable in an aqueous environment, and
wherein said nanodevice comprises biocompatible materials in a composition thereof and has a maximum dimension of less than about 1 μm and greater than about 50 nm.

33. A nanodevice according to claim 32, wherein said nanodevice has a maximum dimension of less than about 400 nm and greater than about 50 nm.

34. A nanodevice according to claim 32, wherein said nanodevice has a maximum dimension of less than about 300 nm and greater than about 50 nm.

35. A nanodevice according to claim 32, wherein said nanodevice has a maximum dimension of less than about 150 nm and greater than about 50 nm.

36. A nanodevice according to claim 32, wherein said valve assembly is operable to at least one of open and close in response to a change of pH in a local environment of said valve assembly.

37. A nanodevice according to claim 32, wherein said valve assembly is operable to open in response to a change to an acidic local environment and to close in response to a change to a non-acidic local environment of said valve assembly.

38. A nanodevice according to claim 32, wherein said nanodevice consists essentially of biocompatible materials in a composition thereof.

39. A nanodevice according to claim 32, wherein said containment vessel comprises silica in a material thereof.

40. A nanodevice according to claim 32, wherein said containment vessel is a mesoporous silica nanoparticle defining a plurality of substantially parallel pores therein, said storage chamber being one of said plurality of substantially parallel pores.

41. A nanodevice according to claim 32, wherein said valve assembly is at least a portion of one of a [2]rotaxane and a [2]pseudorotaxane supramolecular structure.

42. A nanodevice according to claim 41, wherein said at least said portion of one of said [2]rotaxane and said [2]pseudorotaxane comprises a cucurbituril molecule as a moving valve component thereof.

43. A nanodevice according to claim 41, wherein said at least said portion of one of said [2]rotaxane and said [2]pseudorotaxane comprises a cyclodextrin molecule.

44. A nanodevice according to claim 32, further comprising a plurality of anionic or electrostatic molecules attached to an outer surface of said containment vessel,
wherein said anionic or electrostatic molecules provide hydrophilicity or aqueous dispersability to said nanodevice and are suitable to provide repulsion between other similar nanodevices.

45. A nanodevice according to claim 44, wherein said plurality of anionic molecules comprise a phosphonate moiety.

46. A nanodevice according to claim 44, wherein said plurality of anionic molecules are trihydroxysilylpropyl methylphosphonate.

47. A nanodevice according to claim 32, further comprising folate ligands attached to said containment vessel.

48. A nanodevice according to claim 32, further comprising a nanoparticle of magnetic material formed within said containment vessel of said nanodevice.

49. A nanodevice according to claim 48, wherein said nanoparticle of magnetic material is an iron oxide nanoparticle.

50. A nanodevice according to claim 32, further comprising a nanoparticle of gold formed within said containment vessel of said nanodevice.

51. A composition of matter, comprising:
a plurality of nanoparticles, each defining a storage chamber therein; and
a guest material contained within said storage chambers defined by said nanoparticles, said guest material being substantially chemically non-reactive with said nanoparticles,
wherein each nanoparticle of said plurality of nanoparticles has a valve assembly to allow said guest material contained within said storage chambers to be selectively released, and
wherein each nanoparticle of said plurality of nanoparticles comprises biocompatible materials in a composition thereof and has a maximum dimension of less than about 1 μm and greater than about 50 nm.

52. A composition of matter according to claim 51, wherein said valve assembly is operable to at least one of open and close in response to a change of pH in a local environment of said valve assembly.

53. A composition of matter according to claim 51, wherein said valve assembly is operable to open in response to a change to an acidic local environment and to close in response to a change to a non-acidic local environment of said valve assembly.

54. A composition of matter according to claim 51, wherein each nanoparticle of said plurality of nanoparticles comprises silica in a material thereof.

55. A composition of matter according to claim 51, wherein each nanoparticle of said plurality of nanoparticles is a mesoporous silica nanoparticle defining a plurality of substantially parallel pores therein, said storage chamber being one of said plurality of substantially parallel pores.

56. A composition of matter according to claim 51, wherein said valve assembly is at least a portion of one of a [2]rotaxane and a [2]pseudorotaxane supramolecular structure.

57. A composition of matter according to claim 56, wherein said at least said portion of one of said [2]rotaxane and said [2]pseudorotaxane comprises a cucurbituril molecule.

58. A composition of matter according to claim 51, wherein each nanoparticle of said plurality of nanoparticles comprises a surface coating of a hydrophilic group.

59. A composition of matter according to claim 51, wherein each nanoparticle of said plurality of nanoparticles comprises folate ligands attached thereto.

60. A method of administering at least one of a biologically active substance, a therapeutic substance, a neutraceutical substance, a cosmetic substance or a diagnostic substance, comprising:
administering a composition to at least one of a person, an animal, a plant, or an organism, said composition comprising nanoparticles therein, wherein said nanoparticles contain said at least one of biologically active substance, therapeutic substance, neutraceutical substance, cosmetic substance or diagnostic substance therein; and
selectively opening a valve in each of said nanoparticles to allow said at least one of said biologically active substance, therapeutic substance, neutraceutical substance, cosmetic substance or diagnostic substance to escape from said nanoparticles.
